(19) United States
(12) Patent Application Publication
(10) Pub. No.: US 2007/0288024 A1
(43) Pub. Date: Dec. 13, 2007

(54) Bone fixation

(76) Inventor: Sohrab Gollogly, Carmel by the Sea, CA (US)

Correspondence Address: ROBERT D. VARITZ, P.C., 4915 SE 33RD PLACE, PORTLAND, OR, 97202, US

(21) Appl. No.: 11/447,652

(22) Filed: Jun. 6, 2006

(51) Int. Cl. A61B 17/58

(52) U.S. Cl. 606/73

(57) ABSTRACT

A bone fixation method including: (a) depth-penetrating a bone with a bone-fixation device, (b) by such depth-penetrating, establishing a condition of selectively variable, inclined-surface frictional contact between the device and the bone, where the magnitude of friction in such contact is variable in relation to the depth of bone penetration by the device, and (c) following the establishing step, producing a bone-ingrowth anchoring interface between the device and the bone. Also disclosed is an implantable, plural-interactive-mechanism, bone-fixation device which is constructed to employ collectively, when in place in relation to an associated bone, cooperative (a) tapered wedging, (b) bone-material displacement, and (c) promoted bone ingrowth, as bone-anchoring mechanisms.

### BACKGROUND AND SUMMARY OF THE INVENTION

This invention relates to bone fixation, and in particular, to a novel bone-fixation device and to a related methodology.

While the device and methodology of the present invention are employable with respect to bone fixation associated with a number of different bone sites in the human body, one preferred embodiment of the invention is pictured and described herein, simply for illustration purposes, in the setting of bone fixation to the pelvis in a procedure involving the application of corrective, distractive positioning forces applied between the pelvis and a rib in the ribcage, as, for example, to address a scoliosis spinal curvature problem.

As discussed herein, bone fixation involves the implanting of an anatomically compatible mechanical device which is anchored to a selected bone for the purpose of (a) remaining in the anatomy, (b) becoming, as much as possible, “as one” with the associated bone, and (c) providing a secure and stable reaction point for the application of a medically determined positioning/correcting anatomical force within the body skeletal structure. As such, it is, of course, extremely important that bone fixation take hold, so-to-speak, with a high level of both immediate and lasting, and even growing stability, that it be relatively simple and as inexpensive as possible in construction and installation, and that it offer the opportunity for versatility of design without compromising fixation force-application utility, to accommodate both attachment to different kinds and shapes of bone sites, and as well to accommodate different natures of corrective, anatomical force applications. Additionally, installation of a bone-fixation device should be something which is relatively easily performed with substantial confidence that a “single” attachment surgical procedure will work well, and that such a procedure will reliably create an easily and quickly stabilized condition of fixation.

To address these various considerations, the bone-fixation device of the present invention features preferably an elongate, gently fully tapered (preferably frustro-conical) body, or body region, which converges, in relation to this region's tapered form, and along the device's long, or drive, axis, toward one end which acts as the bone-insertion end in the device.

In this preferred embodiment, a length of the outer surface of the tapered region in the device is prepared with a suitable form of “immediate” mechanical anchoring structure, such as screw threads. This anchoring structure may also take other forms, such as one or more elongate, outward projections formed on the inclined, outer, lateral surface, or side, of the device, where this (or these) projection(s) lie(s) along a line (or lines) disposed obliquely relative to the device's long, drive axis. Such a projection may be viewed, effectively, as being part of (i.e., an incremental part of), or having the form of a portion of, a screw thread. Preferably, the included mechanical anchoring structure is one which promotes depth-penetrating, or depth-advancing, of the bone-fixation device into a selected bone via what is recognized herein as being helical relative-motion interaction between the bone and the device—such motion coming about as a consequence of a rotation of the device about its long, drive axis relative to the bone.

Further, the device of the invention is preferably prepared/treated/etc. with an appropriate bone-ingrowth-promoting structure, or substance, which encourages, over time, and after device installation, a condition of bone-growth uniting, or bridging, between a subject bone and the attached/anchored device.

From one procedural point of view (there are many which will be mentioned herein), the invention contemplates the pre-preparation at a selected bone site of an elongate, drilled, tapered bore, or cavity, which has a taper that is intended substantially to match that of the tapered, elongate body region of the proposed fixation device, whereby the device, after preparation of such a bore, may appropriately, quickly, and easily be inserted in a wedging manner into this bore, and rotated suitably about its long axis (the mentioned drive axis) to establish an immediate screw-thread-type anchoring fixation between the device and the subject bore and bone.

The presence and use of substantially matching tapers, as has just been mentioned, also promotes a relatively quickly established high-friction, conical-surface, frictional-binding condition between an inserted device and a matching bore, and the presence and use further of a bone-ingrowth-promoting structure, or substance, on the surface of the inserted device functions importantly to establish eventual bone-ingrowth bridging-anchoring at the interface between the device and the subject bone. The condition of frictional contact (binding) just mentioned is also referred to herein as being a condition of selectively variable, inclined-surface frictional contact, where the magnitude of friction in this contact is variable in relation to the depth of bone penetration by the device, as determined by helical-motion advancing of the fixation device into the bone. Where the bone-ingrowth-promoting structure resides in the resulting interface of frictional contact, referred to as a bone-ingrowth anchoring interface, a bone-ingrowth connecting bridge builds in time between the subject bone and the anchored device.

Naturally, all of the material, or materials employed in the fixation device are chosen to be appropriately compatible with the anatomy so as to avoid rejection. As those skilled in the art know there are many suitable materials currently available for this purpose, none of which materials, per se, forms any part of the present invention. For the purpose of illustration herein, a bone fixation device made in accordance with the features of the invention is fabricated of titanium.

These and other special characteristics and advantages which are offered and attained by the present invention will now become more fully apparent as a preferred embodiment thereof, preferred and manner of implementing and practicing the invention, are described below in conjunction with the accompanying drawings.

### DESCRIPTION OF THE DRAWINGS

FIG. 1 is a very simplified, fragmentary, schematic illustration of a specialized force-application installation of apparatus designed to address a spinal deformity, utilizing force-applying mechanism, the lower end of which in this figure is anchored to the iliac buttress portion of a patient's pelvis utilizing a bone-fixation device made in accordance with one preferred form of the present invention. The particular form of fixation device generally shown in this figure is one which has been equipped, at its exposed upper end in accordance with a ready adaptability characteristic of the present invention, with a ball-like structure which forms, as illustrated in FIG. 1, part of a universal-motion-type ball-and-socket interconnection between this fixation device and certain overhead, direct force-applying structure, the upper end of which is anchored conventionally to an “overhead” rib as generally shown.

FIG. 2 is an enlarged, somewhat more detailed, fragmentary and exploded view showing details of the bone-fixation device which is pictured more generally at the lower side of FIG. 1. In FIG. 2, this device is pictured removed from a suitably prepared, tapered receiving bore in the iliac buttress and, essentially, as illustrated “readied” for insertion and fixation in that bore.

FIG. 3 is a view which is somewhat similar to FIG. 2, but here showing the bone-fixation device of FIG. 2 inserted in the mentioned bone-prepared bore of FIG. 2.

FIG. 4 illustrates, in a fragmentary way, a modified bone-fixation device which includes anchor, or anchoring, structure taking the form of a pair (though more or less in number could be used) of elongate, lateral projections existing on the outside inclined surface of the device.

In none of the drawing figures are the component features of the invention, or of other structure, as well as of the anatomy, shown in necessarily correct proportions and to scale.

### DETAILED DESCRIPTION OF THE INVENTION

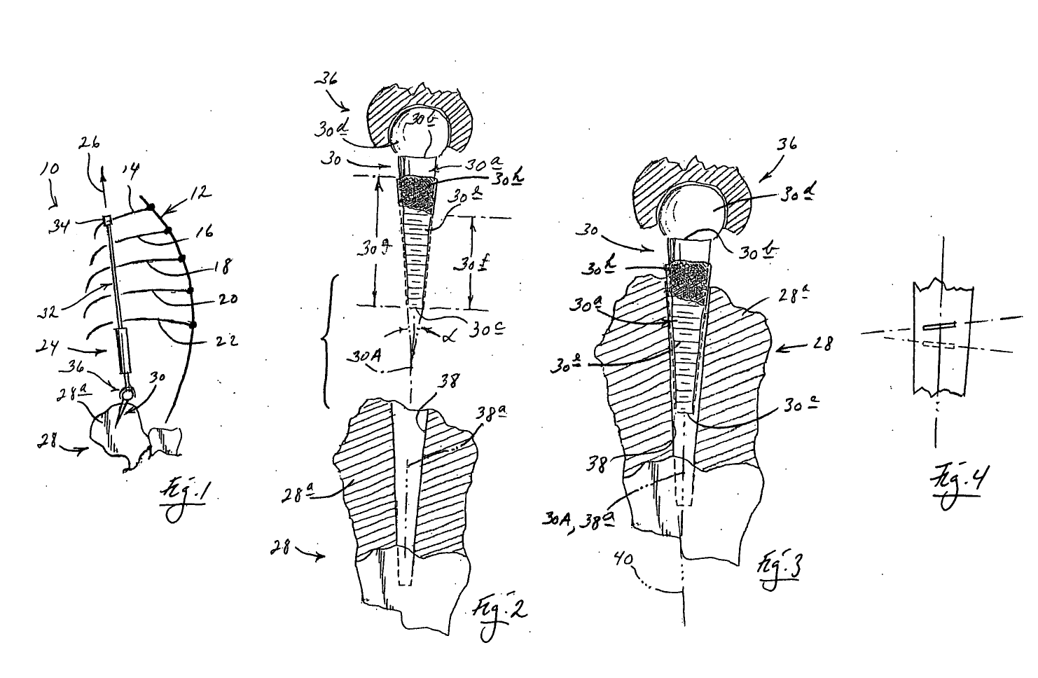

Turning now to the drawings, and referring first of all to FIG. 1, here there is indicated generally at 10 a surgically implemented force-application installation of mechanism designed to correct a spinal deformity which is represented by the arched, highly schematically depicted portion 12 of a patient's spine. Extending to the left in FIG. 1 from spine 12 are five ribs which are shown schematically, and in very simple manners, at 14, 16, 18, 20, 22.

This mechanism, which is shown at 24, is intended to produce a corrective force represented simply by an arrow 26, between fragmentarily illustrated iliac buttress portion 28a of pelvis 28 and rib 14. Included, in a sense, in mechanism 24, which may be otherwise entirely conventional in construction, overall are a bone-fixation device 30 made in accordance with one preferred embodiment of the present invention, an active, elongate, force-application bone-engaging device 32 of any suitable design which forms no part of the present invention, a clamp 34 which clamps the upper operative end of device 32 appropriately to a selected location on rib 14, and a universal-type, ball-and-socket, connectable/disconnectable interconnection 36 which couples the lower, “socket” end of device 32 to the upper, “exposed”, ball-like end of fixation device 30.

It should be understood that, while device 30 is illustrated herein formed with an included ball-like upper end which functions as what is referred to herein as one type of an external-device attaching feature, other suitable types of upper end “attaching feature” structure may also be created un the device to accommodate attachment to other kinds of external devices.

In a manner which will be more fully described shortly, the installed or implanted force-application hardware mechanism which is pictured generally in FIG. 1 is implanted, so-to-speak, within the anatomy during, except for the installation of bone-fixation device 30, an otherwise completely conventional surgical procedure to apply correction forces to deal with the spinal deformity pictured schematically in FIG. 1.

Turning attention now to FIGS. 2 and 3 along with FIG. 1, bone-fixation device 30 which, as mentioned earlier herein, may be made of any suitable anatomically compatible material such as titanium, includes an elongate body 30a which is tapered from its upper to its lower ends in FIGS. 2 and 3, as illustrated, with an included conical angle α (shown in FIG. 2) of about 8-degrees, with this tapered body 30a having upper and lower ends 30b, 30c, respectively. The long axis, also referred to as the drive axis, of body 30a is shown in FIGS. 2 and 3 at 30A. Upper end 30b herein joins integrally with a formed ball-like structure 30d which has been constructed to form part of ball-and-socket connection 36. This ball-like structure (previously generally mentioned) 30d is suitable for enabling a universal-joint-type, relative-motion connection with a device such as force-application device 32, but it should be stated again, and understood, that the uppermost portion of fixation device 30, adjacent the upper end of body 30a, may be formed with any suitable attaching-feature structure designed for connection to some appropriate external structure. Accordingly, ball-like structure 30d is illustrative only herein.

Formed, along with what is referred to herein as an end stretch in the body, on the outside of tapered body 30a are screw threads 30e which are referred to herein as bone anchor, or anchoring, structure. These threads are distributed along a length portion 30f in body 30a.

Effectively distributed along a length portion 30g in body 30a, and longitudinally overlapping screw threads 30e as seen in FIGS. 2 and 3, is what is referred to herein as an established bone-ingrowth-promoting zone 30h. This zone may be formed in any appropriate conventional manner, and in the embodiment of device 30 shown herein, zone 30h is formed of sintered titanium.

Iliac buttress portion 28a of pelvis 28 is prepared, in accordance with practice of the present invention, with an elongate tapered bore, or cavity, 38 having an inwardly and downwardly (in FIGS. 2 and 3) converging taper which substantially matches the taper formed in fixation device body 30a. The long axis of bore 38 is shown in FIGS. 2 and 3 at 38a. Preferably, bore 38, which is also referred to herein as a fixation-reception cavity, is prepared in such a fashion that when device 30 is seated by grip-producing insertion, and anchored, in this bore, the upper portion of the device projects a short distance above the upper end of the bore. This condition is illustrated very clearly in FIG. 3 (and also in FIG. 1).

In the practice of the invention, device 30 is sized appropriately to fit at a predetermined location in the skeletal structure of the human body, with the tapered body portion 30a of this device being designed with an appropriate length. In the particular illustration now being described, the full axial length of body portion 30a in device 30 is about 2-inches. Threads 30e are distributed along body 30a for the previously mentioned length 30f which, herein, is about 1½ inches. Bone-ingrowth-promoting zone 30h herein substantially fully covers threads 30e, and extends along body 38, in total, for a length 30g of about 1¾-inches.

With a bore, such as bore 38, prepared appropriately, body 30a in device 30 is inserted into this bore as a part of the associated surgical procedure, and is turned (rotated) about its drive axis 30A to cause screw threads 30e to bite into the conical wall of bore 38. The result of this action is helical relative motion between the device and the bone, with the device, as a consequence, depth-penetrating, or depth advancing, into the bone. Biting in, and bone-material displacement by, the action of, these threads, along with a relatively broad-surface-contact wedging action which occurs because of the tapered natures of body 30a and bore 38, causes device 30 to become extremely securely anchored in place in iliac buttress 28a. This condition is referred to herein as one of selectively variable, inclined-surface (see dash-triple-dot line 40 in FIG. 3), frictional contact between the device and the bone, wherein the magnitude of friction is variable in relation to the depth of bone penetration by the device. With this condition existing, as illustrated for example in FIG. 3, bone-ingrowth-promoting zone 30h confronts the tapered wall of bore 38, and provides a very effective bone-ingrowth anchoring interface for promoting bone-ingrowth bridging (i.e., the building of a bone bridge) in relation to device 30.

FIG. 4 in the drawings pictures, fragmentarily, a modified bone-fixation device is shown at 42, including an elongate, tapered body 42a, and a long, drive axis 42A. Device 42 differs from device 30 principally by possessing, as mechanical bone anchor, or anchoring, structure, a pair of elongate, lateral outward projections 44, 46 whish, as illustrated herein, are positioned on opposite lateral sides of body 42a. These projections lie, respectively, along lines 44a, 46a which lie obliquely relative to axis 42A. Projections 44, 46 herein each forms, effectively, an incremental portion of a right-hand screw thread, and with turning of device 42 about its drive axis 42A, and when engaged with an appropriately tapered wall of a prepared cavity, or bore, such as cavity 38, what takes place is helical relative motion of device 42 relative to the associated bone, with resulting depth-penetrating of the device into the bone.

Accordingly, a uniquely formed bone-fixation device, featuring a tapered, long, bone-insertion body bearing outside mechanical anchoring structure, such as screw threads, or the like, and bone-ingrowth-promoting structure, has been illustrated and described herein. This device can be viewed generally as taking the form of an implantable, plural-interactive-mechanism, bone-fixation device which is constructed to employ collectively, when in place in relation to an associated bone, cooperative (a) tapered wedging, (b) bone-material displacement, and (c) promoted bone ingrowth, as bone-anchoring mechanisms.

So also described is a related, unique bone-fixation methodology which, in a somewhat specific and more detailed point of view, can be characterized generally as including the steps of (a) creating an elongate, generally at least partially conically tapered fixation-reception cavity in a selected bone, (b) preparing, adjacent one end of an elongate bone-fixation device, an end stretch possessing a generally, at least partially conical end-stretch taper which is complementary to the conical taper of the created cavity, and in functional association with that end stretch, also preparing exposed anchor structure which is suitable for grip-producing insertion in the created reception cavity, (c) establishing on the outside of the prepared end stretch, in association with the anchor structure, a bone-ingrowth-promoting zone, and (d) utilizing the device's anchor structure, inserting the prepared end stretch into the created cavity in a manner which produces a grip condition between the device and the cavity, and which also disposes at least a portion of the bone-ingrowth-promoting zone within the cavity.

The disclosed device and methodology of the invention, viewed in all of the several ways suggested above, offer significant improvements in the surgical implantation of bone-fixation (and associated) hardware. This device and methodology offer, for example, a great deal of flexibility and versatility in the practice, generally, of surgical bone fixation, and as suggested above, the features of the invented device, and of the associated methodology, are readily adaptable, through modification, to various, different, bone-fixation applications where different device sizes, and differently sized receiving bores, may be most appropriate. The angle of “taper” is also freely choosably variable, as is the selected nature of the tapered-body-included “anchoring structure”—perhaps other than screw threads, or lateral, outward projections. Bone-ingrowth-promoting may also be accomplished other than through the use of sintered titanium.

Still another kind of modification might involve forming a true conically tapered surface, per se, only at a few angularly-displaced locations, relative to the long axis of the device body, on the outside of that body—i.e., preparing what is referred to herein as a generally, at least partially conical end-stretch taper in the body of the device. Under such a modified approach, angularly intermediate (non-conical) regions might, for examples, be flattened or inwardly grooved (or channeled) to minimize material content in the body, or perhaps for other useful reasons.

Still another modification of the invention may take the form of a bone-fixation device, wherein the mechanical anchor structure, and/or the bone-ingrowth-promoting structure, need not necessarily be formed or provided on the conical portion of the body in the device. Those skilled in the relevant art, from the just-made comment about this kind of relative positioning (operative association/relation type) modification, will readily appreciate that there are many ways to achieve such structure within the context of the present invention.

As was also mentioned earlier, the bone-fixation device of this invention may be made from a variety of freely selectable, anatomically compatible materials.

Accordingly, while preferred embodiments of the present invention, and preferred practices of associated methodology, have been described and illustrated herein, with certain possible variations and modifications suggested, it is appreciated that other variations and modifications may be made without departing from the sprit of the invention.

I claim:

1. A bone-fixation method comprising
depth-penetrating a bone with a bone-fixation device, and thereby establishing a condition of selectively variable, inclined-surface frictional contact between the device and the bone, where the magnitude of friction in such contact is variable in relation to the depth of bone penetration by the device, and
following said depth-penetrating and establishing, producing a bone-ingrowth anchoring interface between the device and the bone.

2. The method of claim 1, wherein said producing is performed at a location at least partially coincident with the region of inclined-surface frictional contact.

3. A bone-fixation method comprising
using generally helical relative motion, depth-advancing a bone-fixation device into a bone,
by said advancing, establishing a condition of inclined-surface compression contact between the device and the bone, where the level of compression in the region of device/bone inclined-surface contact is related to the depth of advancement of the device into the bone, and
following said establishing, producing a bone-ingrowth connective bridge between the device and the bone in the region of inclined-surface compression contact.

4. The method of claim 3, wherein the bone-fixation device has a long axis, and an elongate, lateral outward projection which generally extends along a line disposed at an oblique angle relative to the device's long axis, and said using and advancing steps involve relative rotational contact between the bone and the device's outward projection.

5. The method of claim 4, wherein the device's elongate, lateral outward projection has the form of a part of a screw thread.

6. A bone-fixation method comprising
utilizing helical relative motion between a bone and a bone-fixation device of a type having a drive axis, driving the devise into the bone substantially along the mentioned drive axis,
by said driving, and relative to the device's drive axis, establishing a condition of inclined-surface frictional contact between the device and the bone, and
following said establishing, producing a bone-ingrowth anchoring interface between the device and the bone.

7. A bone-fixation method comprising
creating an elongate, generally conically tapered, fixation-reception cavity in a selected bone,
preparing, adjacent one end of an elongate bone-fixation device, an end stretch possessing a generally, at least partially conical end-stretch taper which is complementary to the conical taper of the created cavity, and in functional association with that end-stretch taper, also preparing exposed anchor structure which is suitable for grip-producing insertion in the created reception cavity,
establishing on the outside of the prepared end stretch, in association with the anchor structure, a bone-ingrowth-promoting zone, and
utilizing the device's anchor structure, inserting the prepared end stretch into the created cavity in a manner which produces a grip condition between the device and the cavity, and which also disposes at least a portion of the bone-ingrowth-promoting zone within the cavity.

8. The method of claim 7, wherein said preparing of an anchor structure includes the forming of screw threads.

9. The method of claim 7, wherein the established bone-ingrowth-promoting zone includes a portion of the prepared anchor structure.

10. The method of claim 9, wherein said preparing of an anchor structure includes the forming of screw threads.

11. The method of claim 7 which further comprises providing the end of the bone-fixation device which is opposite the device's mentioned one end with an external-device attaching feature which is suitable for operative attachment to an external bone-engaging device.

12. The method of claim 11, wherein the external-device attaching feature takes the form generally of a connection sub-structure which functions as a component in a universal type joint for operative attachment to an external bone-engaging device.

13. The method of claim 12, wherein the mentioned connection sub-structure takes the form generally of a ball which acts as a component in a ball-and-socket type structure.

14. An implantable, plural-interactive-mechanism, bone-fixation device which is constructed to employ collectively, when in place in relation to an associated bone, cooperative (a) tapered wedging, (b) bone-material displacement, and (c) promoted bone ingrowth, as bone-anchoring mechanisms.

15. A bone fixation device comprising
an elongate body,
an at least partially conically tapered, elongate stretch in said body extending convergingly toward one end of the body,
exposed bone anchor structure formed in operative relation to said stretch,
a bone-ingrowth-promoting zone also formed in operative relation to said stretch, and in operative association with said anchor structure, and
an external-device attaching feature formed in said body adjacent its other end.

16. The device of claim 15, wherein said bone anchor structure is formed on said stretch.

17. The device of claim 15, wherein said bone-ingrowth-promoting zone is formed on said stretch.

18. The device of claim 15, wherein said bone anchor structure said bone-ingrowth-promoting zone are formed on said stretch.

19. The device of claim 15, wherein said attaching feature takes the form of a ball suitable for use as a component in a ball-and-socket type universal joint.

20. The device of claim 15, wherein said stretch is fully conically tapered along its length.

21. The device of claim 15, wherein said bone anchor structure takes the form of screw threads.

22. The device of claim 21, wherein said attaching feature takes the form of a ball suitable for use as a component in a ball-and-socket type universal joint.
